(19) United States
(12) Patent Application Publication
(10) Pub. No.: US 2018/0303867 A1
(43) Pub. Date: Oct. 25, 2018

(54) SUPPRESSION OR REDUCTION OF THE PATHOGENICITY OR VIRULENCE OF A CLOSTRIDIUM BACTERIA

(71) Applicant: Da Volterra, Paris (FR)

(72) Inventors: Jean De Gunzburg, London (GB); Mark Wilcox, Leeds (GB); Caroline Chilton, Leeds (GB)

(21) Appl. No.: 15/765,449

(22) PCT Filed: Sep. 30, 2016
(86) PCT No.: PCT/EP2016/073526

§ 371 Date: Apr. 2, 2018

(30) Foreign Application Priority Data
Oct. 6, 2015 (EP) 15306577.6

(51) Int. Cl. A61K 31/731; A61K 33/44; A61P 31/04

(52) U.S. Cl. CPC A61K 31/731; A61P 31/04; A61K 33/44

(57) ABSTRACT

The invention relates to the use of an adsorbent to reduce or suppress the pathogenicity or virulence of Clostridium bacteria in vitro, ex vivo or in vivo. The invention may be used in any mammal or environment, and is particularly effective to suppress the pathogenicity or virulence of Clostridium difficile.

### FIELD OF THE INVENTION

The invention relates to the use of an adsorbent to reduce or suppress the pathogenicity or virulence of Clostridium bacteria in vitro, ex vivo or in vivo. The invention may be used in any mammal or environment, and is particularly effective to suppress the pathogenicity or virulence of Clostridium difficile.

### BACKGROUND OF THE INVENTION

Clostridium difficile pertains to a particular subset of bacterial pathogens of concern: spore-forming bacteria. Bacterial spores (endospores) are dormant, non-reproductive structures formed by bacteria in response to environmental stress. Once environmental conditions become favorable, the spores germinate and the bacteria proliferate. In the case of pathogenic bacteria, germination in a human host may result in disease. Germination of a spore, such as Clostridium difficile, is the process in which the spore gives birth to a vegetative bacterium that is then able to grow and multiply. Bacterial spores are extremely tolerant to many agents and environmental conditions including radiation, desiccation, temperature, starvation and chemical agents. This natural tolerance to several agents allows spores to persist for many months in key environments such as hospitals, other healthcare centers and food production facilities, where standard cleaning agents, germicides and sterilization processes do not eradicate the bacteria. In the case of food production, the presence of spores can have significant consequences ranging from simple food spoilage to the spread of food-borne pathogens and food poisoning.

Members of the genus Clostridium are Gram-positive, spore-forming, obligate anaerobes. Exemplary species causing human disease include, but are not limited to, C. perfringens, C. tetani, C. botulinium, C. sordellii and C. difficile. Clostridia are associated with diverse human diseases including tetanus, gas gangrene, botulism and pseudomembraneous colitis and can be a causative agent in food poisoning. Of particular concern is the disease caused by Clostridium difficile. Clostridium difficile causes Clostridium difficile-associated disease (CDAD), also termed Clostridium difficile infection (CDI), and there has been a ten-fold increase in the number of cases within the last 10 years all over the world, with hyper-virulent and drug resistant strains now becoming endemic.

Clostridium difficile is a commensal enteric bacterium, the levels of which are kept in check by the normal gut flora. However, the bacterium is the causative agent of C. difficile associated disease (CDAD) and has been identified as the primary cause of the most serious manifestation of CDAD, pseudomembraneous colitis. CDAD is associated with a wide range of symptoms ranging from mild diarrhea to serious bloody diarrhea, pseudomembraneous colitis, toxic megacolon and death. The primary risk factor for the development of CDAD is the use of antibiotics disrupting the normal enteric bacterial microbiota enabling an overgrowth of Clostridium difficile. Although clindamycin has been one of the first major antibiotics associated with CDAD, the disease occurs following the treatment of patients with nearly all antibiotics including members of the fluoroquinolone, lincosamide, β-lactam (including penicillins, cephalosporins and carbapenems, whether given alone or together with β-lactamase inhibitors) and many others classes. There is a strong literature describing the increased odds ratio of acquiring a CDAD after antibiotic treatment. Recent communication from the US CDC states that “people on antibiotics are 7-10 times more likely to get C. difficile [infections] while on the drugs and during the month after”. Nowadays we also see many cases of CDAD being reported with no underlying antibiotic use, especially in case of C. difficile outbreaks in healthcare facilities.

CDAD is primarily of concern in the hospital setting and is of particular concern amongst elderly patients where mortality rates are particularly high. Mortality rates in the USA have risen from 5.7 per million of population in 1999 to 23.7 per million in 2004. Colonization rates of C. difficile in the general population are up to 3% although hospitalization dramatically increases the rates of colonization up to 25%. Of particular concern is the emergence of new endemic strains. A particularly relevant example are the hyper-virulent BI/NAP1 (also known as ribotype 027) strains which show increased toxin A (TcdA) and toxin B (TcdB) production, as well as the production of an additional toxin termed as binary toxin. The hyper-sporulation characteristics of strains such as the hypervirulent strains of ribotype 027 contribute significantly to the issue.

The two major toxins produced by toxigenic strains of C. difficile, TcdA and TcdB, have been studied intensively since their initial recognition as major C. difficile virulence factors. C. difficile toxins A (TcdA) and B (TcdB) are encoded by two separate but closely linked genes that together form part of a 19.6 kilobase region known as the “toxigenic element” or the “pathogenicity locus.” TcdA and TcdB genes and proteins are highly homologous, and it is likely that the genes evolved by duplication. Toxins A and B are produced simultaneously in most C. difficile toxigenic strains; they are responsible for the pathogenicity of Clostridium difficile. The toxins begin to be produced during the exponential growth phase, and they are usually released from the bacteria between 36 and 72 hours of culture, when the bacteria have reached the stationary phase. Toxins present within the bacteria can be released earlier by sonication or by use of a French pressure cell.

Early works demonstrated a direct relationship between toxin levels and development of pseudomembranous colitis and duration of diarrhea (Burdon, D. W., R. H. George, G. A. Mogg, Y. Arabi, H. Thompson, M. Johnson, J. Alexander-Williams, and M. R. Keighley. 1981. Faecal toxin and severity of antibiotic-associated pseudomembranous colitis. J. Clin. Pathol. 34:548-551.). More direct evidence for the role of TcdA in C. difficile-associated disease comes from studies in gnotobiotic mice showing protection against disease with monoclonal antibodies to TcdA (Corthier, G., M. C. Muller, T. D. Wilkins, D. Lyerly, and R. L'Haridon. 1991. Protection against experimental pseudomembranous colitis in gnotobiotic mice by use of monoclonal antibodies against Clostridium difficile toxin A. Infect. Immun 59:1192-1195.). It has also recently been shown that neutralization of C. difficile toxins with an anionic high-molecular-weight polymer protected 80% of experimental hamsters exposed to the organism (Kurtz, C. B., E. P. Cannon, A. Brezzani, M. Pitruzzello, C. Dinardo, E. Rinard, D. W. Acheson, R. Fitzpatrick, P. Kelly, K. Shackett, A. T. Papoulis, P. J. Goddard, R. H. Barker, Jr., G. P. Palace, and J. D. Klinger. 2001. GT160-246, a toxin binding polymer for treatment of Clostridium difficile colitis. Antimicrob. Agents Chemother. 45:2340-2347.).

The respective roles and contributions of TcdA and TcdB to the disease are not well understood. Early work had suggested that the hallmarks of pseudomembranous colitis, including fluid accumulation, inflammation, and cell damage, can be induced with TcdA in animal models (D. M. Lyerly et al, Infect. Immun 54:70-76, 1986).

However, this result has since been challenged in several reports. In fact, an early study found that TcdB was unable to initiate disease unless TcdA was present (Lyerly, D. M., K. E. Saum, D. K. MacDonald, and T. D. Wilkins. 1985. Effects of Clostridium difficile toxins given intragastrically to animals. Infect. Immun 47:349-352). Naturally occurring TcdA− TcdB+ strains are occasionally identified from clinical isolates (Sambol, S. P., M. M. Merrigan, D. Lyerly, D. N. Gerding, and S. Johnson. 2000. Toxin gene analysis of a variant strain of Clostridium difficile that causes human clinical disease. Infect. Immun 68:5480-5487) and have been useful in characterizing the role of TcdB in C. difficile-associated disease. Interestingly, these strains are capable of causing disease, and in some cases of extensive pseudomembranous colitis, patients died from disease caused by a TcdA-deficient strain (Alfa, M. J., A. Kabani, D. Lyerly, S. Moncrief, L. M. Neville, A. Al-Barrak, G. K. Harding, B. Dyck, K. Olekson, and J. M. Embil. 2000. Characterization of a toxin A-negative, toxin B-positive strain of Clostridium difficile responsible for a nosocomial outbreak of Clostridium difficile-associated diarrhea. J. Clin. Microbiol. 38:2706-2714). More recently, it has been suggested that both TcdA and TcdB play important roles in the disease (S. A. Kuehne, S. T. Cartman, J. T. Heap, M. L. Kelly, A. Cockayne and N. P. Minton. 2010. The role of toxin A and toxin B in Clostridium difficile infection. Nature 467:711-713, 2010); yet two other well documented reports based on experiments in several animal models suggest that the principal factor in the virulence of Clostridium difficile and contribution to the disease is TcdB (D. Lyras, J. R. O'Connor, P. M. Howarth, S. P. Sambol, G. P. Carter, T. Phumoonna, R. Poon, V. Adams, G. Vedantam, S. Johnson, D. N. Gerding, J. I. Rood. 2009. Toxin B is essential for virulence of Clostridium difficile. Nature 458:1176-1179. 10.1038/nature07822.).

The primary therapy option for the treatment of CDAD is discontinuation of any current antimicrobial treatment followed by appropriate use of either vancomycin or metronidazole. Both agents are usually administered orally although metronidazole may also be administered intravenously and in severe cases, vancomycin may also be administered via numerous other routes including intracolonic, through nasal gastric tube or as a vancomycin-retention enema. Additional antibiotic agents that have been reported to be used in the treatment of CDAD include fidaxomicin, fusidic acid, rifamycin and its analogues, teicoplanin and bacitracin, although none show particular efficacy over vancomycin or metronidazole to cure the primary infection. However, one of the most daunting problems with CDAD is the frequent recurrence of the disease. Indeed, following treatment with vancomycin or metronidazole, recurrence of the disease within 3 months is observed in 25-30% of cases. But if a patient has already experienced a previous CDAD episode, the recurrence rate is higher (around 40%); it becomes higher and higher after each recurrence such that recurrence becomes inevitable after the 5^{th} or 6^{th} CDAD episode, leaving no other treatment option that a Fecal Microbiome Transplant (FMT). The rate of recurrence is significantly lower following treatment with fidaxomycin, 15% vs 25% (T. J. Louie, M. A. Miller, K. M. Mullane, K. Weiss, A. Lentnek, Y. Golan, S. Gorbach, P. Sears, Y. K. Shue, OPT-80-003 Clinical Study Group. 2011. N Engl J Med 364:422-31) which has been the basis for the great interest in the use of this recent antibiotic. It is believed that the main factor in the high recurrence rate following treatment of CDAD with vancomycin and metronidazole is their profoundly disruptive effect on the gut microbiota; the fact that fidaxomycin has a narrower spectrum of action than vancomycin and metronidazole could contribute to the lower observed recurrence rate following its use. In addition to halting any offending antibacterial treatment, the use of antiperistaltic agents such as opiates, or loperamide should be avoided since they can reduce clearance of the C. difficile toxins and exacerbate toxin-mediated colonic injury.

Alternative therapies, used as stand-alone agents or in conjunction with antibacterials, are aimed at one or several of the following strategies, such as trying to prevent the antibiotic-mediated disruption of the microbiota, re-establish the native gut microorganism population, reducing the levels of C. difficile toxins or stimulating the immune system. Thus, alternative CDAD therapies include provision of Saccharomyces boulardii or Lactobacillus acidophilus in conjunction with antibiotics, but with unconvincing results. In severe cases where all other therapy options have failed, surgery has been used; although rates of colectomy are low (up to 3% of cases) it is associated with high mortality rates (up to 60%). A recent and highly successful option consists in Fecal Microbiome Transplant (FMT) that enables to obtain cure rates above 85% (Hamilton M J, Weingarden A R, Sadowsky M J, et al. Standardized Frozen Preparation for Transplantation of Fecal Microbiota for Recurrent Clostridium difficile Infection. Am J Gastroenterol. 2012; 107:761-767. doi: 10.1038/ajg.2011.482); novel curative options based on the FMT principle are rapidly developing.

Clostridium difficile associated diseases (CDAD) or Clostridium difficile infection (CDI) is the major identifiable cause of antibiotic-associated diarrhea in hospitals (Cohen S, Gerding D, Johnson S, et al. Clinical practice guidelines for Clostridium difficile infection in adults: 2010 update by the Society for Healthcare Epidemiology of America (SHEA) and the Infectious Diseases Society of America (IDSA). Infect Control Hosp Epidemiol 2010; 31:431-55; Kelly C P. Current strategies for management of initial Clostridium difficile infection. J Hosp Med 2012; 7:S5-10). In the USA alone, CDAD has been reported in >500,000 patients per year, with up to 29,000 deaths per year (Rupnik M, Wilcox M H, Gerding D N. Clostridium difficile infection: new developments in epidemiology and pathogenesis. Nat Rev Microbiol 2009; 7:526-36, Lessa et al. Burden of Clostridium difficile Infection in the United States. N Engl J Med 2015; 372:825-834). The yearly healthcare burden has been estimated to be >S3 billion in the United States (Kachrimanidou M, Malisiovas N. Clostridium difficile infection: A comprehensive review. Crit Rev Microbiol 2012; 37:178-87.). A recent study reported that CDAD is responsible for 25% more nosocomial infections than methicillin-resistant Staphylococcusaureus (Voelker R. Increased Clostridium difficile virulence demands new treatment approach. J Am Med Assoc 2010; 303:2017-9.). The rate of CDAD progression to severe symptoms and death has been increasing (Valiquette L, Low D E, Pepin J, McGeer A. Clostridium difficile infection in hospitals: a brewing storm. Can Med Assoc J 2004; 171:27-9.).

There is a pressing need for new and effective agents and treatments to prevent and treat diseases associated with spore-forming bacteria, particularly those caused by members of the genera Clostridium and in particular diseases associated with Clostridium difficile infection. This need is particularly acute in the light of the refractory nature of Clostridium difficile to many broad spectrum antibiotics (including β-lactam and quinolone antibiotics) and the frequency with which resistance emerges and episodes recur.

### SUMMARY OF THE INVENTION

The present invention relates to compositions and methods for treating or preventing Clostridium-associated diseases. The invention more particularly relates to the use of an adsorbent to suppress the pathogenicity of Clostridium bacteria in vitro, ex vivo or in vivo. The invention stems, inter alia, from the unexpected finding by the inventors that adsorbents can effectively alter the virulence of Clostridium bacteria, by inhibiting the production and release of toxins, the sporulation and spore germination of Clostridium, leading to an effective neutralization of the pathogenicity of these bacteria.

An object of the invention thus relates to an adsorbent for use to treat a Clostridium-associated disease in a mammal.

A particular object of the invention relates to an adsorbent for use to prevent a Clostridium-associated disease in a mammal.

A further object of the invention relates to an adsorbent for use to suppress the pathogenicity of a bacterium of the genus Clostridium.

The invention also relates to a method for suppressing the pathogenicity of a bacterium of the genus Clostridium, comprising exposing said bacterium to an adsorbent.

The invention also relates to a method for treating a Clostridium-associated disease in a mammal comprising exposing said mammal to an adsorbent.

The invention also relates to a method for preventing a Clostridium-associated disease in a mammal comprising exposing said mammal to an adsorbent.

The invention also relates to a method for decreasing the severity and mortality of a Clostridium-associated disease in a mammal comprising exposing said mammal to an adsorbent.

The invention also relates to the use of an adsorbent to suppress or delay the production of toxins by a bacterium of the genus Clostridium, or to suppress or delay the germination of spores of a bacterium of the genus Clostridium, and/or to suppress or delay the sporulation of a bacterium of the genus Clostridium.

According to preferred embodiments, the adsorbent is an activated charcoal and/or the bacterium is a Clostridium difficile. Also, in a preferred embodiment, the invention is used in vivo in a mammal and the adsorbent is administered orally, preferably with a formulation that releases the adsorbent in the gastrointestinal tract, particularly in the lower part of the small intestine, particularly at the ilea-caecal junction. Preferably, the formulation releases the adsorbent in the late ileum, i.e., just before the caecum and colon. The invention may be used in any mammal, such as human or non-human animals, and may be used in a curative or preventive setting. The invention may also be used to prevent contamination or spreading of Clostridium spores and/or of Clostridium-related diseases in any environment such as hospitals.

### LEGEND TO THE FIGURES

FIG. 1: Counts of C. difficile bacteria, counts of spores and toxin titers in an adsorbent-treated BHI broth after 48 h of culture. Mean counts for C. difficile strains ribotype 001, 027 and 078.

FIG. 2: Counts of C. difficile bacteria, spores and toxin titers in an adsorbent-treated fecal slurry broth after 48 h of culture. Mean counts for C. difficile strains ribotype 001, 027 and 078.

FIG. 3: Counts of C. difficile bacteria, spores and toxin titers in gut an adsorbent-treated gut model broth after 48 h of culture. Mean counts for C. difficile strains ribotype 001, 027 and 078.

FIG. 4: Measure of C. difficile toxin titers in BHI broth for ribotype 027 strain after incubation with activated charcoal.

FIG. 5: Measure of C. difficile toxin titers in BHI broth for ribotype 078 strain after incubation with activated charcoal.

FIG. 6: Measure of C. difficile toxin titers in BHI broth for ribotype VA-11 REA J strain after incubation with activated charcoal.

FIG. 7: Measure of C. difficile germination in fecal slurry broth for C. difficile strains ribotype 001, 027 and 078 after incubation with activated charcoal.

### DETAILED DESCRIPTION OF THE INVENTION

The present invention relates to compositions and methods for treating or preventing Clostridium-associated diseases and to suppress the pathogenicity of Clostridium bacteria in vitro, ex vivo or in vivo. The invention stems, inter alia, from the unexpected finding that adsorbents can effectively alter the virulence of Clostridium bacteria, by inhibiting the production and release of toxins, the sporulation and/or spore the germination of Clostridium bacteria, leading to an effective neutralization of the pathogenicity of these bacteria and a potent prevention and/or treatment of diseases or conditions caused by such bacteria.

### Adsorbent and Formulations

The term adsorbent designates any compound or material that can adsorb a substrate, typically by physico-chemical binding between the adsorbent surface and the substrate(s) to be adsorbed. Adsorbents may be specific or non-specific. Preferred adsorbents for use in the invention are pharmaceutical grade adsorbents, best suited for use in humans or animals for pharmaceutical or veterinary applications.

Examples of adsorbents suitable for use in the present invention include, without limitation, activated charcoal; clays, including bentonite, kaolin, montmorrillonite, attapulgite, halloysite, laponite, and the like; silica, including colloidal silica (Ludox® AS-40 for example), mesoporous silica (MCM41), fumed silica, zeolites and the like; talc; cholesteramine and the like; polystyrene sulfonates and the like; mono and polysulfonated resins; as well as other resins such as those used for bacteriologic testing such as BACTEC® resins.

Preferred adsorbents are activated charcoals (Chemviron, Cabot, Norit, Merck or other sources) which are pharmaceutical grade. In a particular embodiment, the adsorbent is activated charcoal, more preferably an activated charcoal having a specific surface area above 1000 m²/g, preferentially above 1200 m²/g, preferentially above 1400 m²/g, preferentially above 1600 m²/g, even more preferably above 1800 m²/g. In a preferred embodiment, the activated charcoal is of vegetal origin. In a preferred embodiment, the activated charcoal is characterized by a molasses number greater than 200, preferably greater than 250, more preferably greater than 400 or even greater than 600. In a preferred embodiment, the activated charcoal exhibits a Methylene blue adsorption greater than 20 g/100 g, preferably greater than 30 g/100 g or even more preferably greater than 35 g/100 g.

The amount of adsorbent employed in the methods of the invention may vary depending upon the host/material being treated and the overall capacity, adsorption power and selectivity of the adsorbent. Typically, the amount of adsorbent is an amount sufficient to suppress the pathogenicity of a Clostridium bacterium, or to produce a therapeutic effect, for example a therapeutically significant decrease in the amount of spores and/or toxins released by the bacterium in the terminal parts of the gut, in particular in the colon, or a significant delay in the maturation of the bacteria i.e. the production and further shedding of spores in the environment.

The adsorbents for use in the present invention may be formulated in any acceptable and suitable composition. Such suitable compositions include formulations for oral delivery, rectal delivery, local application, mucosal application, inhalation, and the like. In a particular embodiment, the adsorbent is formulated in a pharmaceutical composition suitable for administration to humans or animals. More preferably, the adsorbent is formulated in an oral formulation adapted to release said adsorbent in the intestine or in contact with intestinal bacteria, particularly in the gastrointestinal tract, more particularly in the lower part of the intestine, i.e. the late ileum, the caecum and/or the colon.

Examples of formulations suitable for intestinal delivery of an adsorbent have been described in WO2006/122835 and WO2007/132022. In a preferred embodiment, the absorbent is formulated with a carrageenan, preferably in the form of a pellet, as proposed in WO2011/104275. Such a formulation can form a core, which may be covered with a layer of a coating such that the adsorbent is released in the lower part of the intestine, i.e., in the late ileum, caecum and/or colon.

Carrageenan is a naturally-occurring family of linear sulphated polysaccharides which are extracted from red seaweeds. Carrageenans are high molecular weight polysaccharides made up of repeating galactose and 3,6-anhydrogalactose (3,6-AG) units, both sulfated and non-sulfated. The units are joined by alternating alpha 1-3 and beta 1-4 glycosidic linkages. Three basic types of carrageenan are available commercially, i.e. kappa, iota, and lambda carrageenans, which differ by the number and position of the ester sulfate groups on the galactose units. The carrageenan for use in the present invention can be selected from kappa, iota and lambda carrageenans, and mixtures thereof. In one aspect of this embodiment, the adsorbent is mixed with kappa-carrageenan. In a particular embodiment, the mixture comprises activated charcoal and kappa-carrageenan.

Preferably, the amount of carrageenan is between about 5% and about 25%, more preferably between about 10% and about 20%, by weight of the mixture of the adsorbent with the carrageenan. According to a specific embodiment of the invention, the amount of carrageenan is about 15% by weight of the mixture. For example, the mixture may contain 85% of an adsorbent and 15% of carrageenan, by weight of the total mixture.

According to a particular embodiment of the invention, a mixture of activated charcoal and carrageenan is provided with the weight ratios indicated above.

The core (or pellet) may be produced by any suitable means known to the skilled artisan. In particular, granulation techniques are adapted to produce said core. For example, the core may be obtained by mixing the adsorbent and the carrageenan in the ratios indicated above, adding a solvent such as water to proceed to wet granulation, followed by extrusion spheronization or one-pot pelletization. Any remaining water can be removed, for example, by drying the resulting pellets using conventional techniques.

In one embodiment, the core, or pellet has an average wet particle size in the range from 250 to 3000 μm, in particular 250 to 1000 μm, in particular 300 to 3000 μm (such as 500 to 3000 μm), in particular 300 to 1000 μm (such as 500 to 1000 μm), in particular 500 to 1000 μm, in particular 500 to 700 μm.

The core composition can further include conventional excipients such as anti-adherents, binders, fillers, diluents, flavours, coloration agents, lubricants, glidants, preservatives, sorbents and/or sweeteners. The amounts of such excipients can vary, but are typically in the range of 0.1 to 50% by weight of the pellet.

As discussed above, a preferred formulation of the invention comprises a core comprising an adsorbent, possibly supplemented with carrageenan, which core is covered with a layer of a coating such that the adsorbent is released in the lower part of the intestine, i.e., in the late ileum, caecum and/or colon.

In this regard, in a preferred embodiment, the adsorbent is used as a formulation comprising:
a core containing the adsorbent and carrageenan, and
a layer of an external coating formed around the core such that the adsorbent is released from the formulation in the lower part of the small intestine.

Examples of suitable coatings include pH-dependent enterosoluble polymers, azopolymers, disulphide polymers, and polysaccharides, in particular amylose, pectin (e.g. pectin crosslinked with divalent cations such as calcium pectinate or zinc pectinate), chondroitin sulphate and guar gum. Representative pH-dependent enterosoluble polymers include cellulose acetate trimellitate (CAT), cellulose acetate phthalate (CAP), anionic copolymers based on methylacrylate, methylmethacrylate and methacrylic acid, hydroxypropyl methylcellulose phthalate (HPMCP), hydroxypropylmethylcellulose acetate succinate (HPMCAS), methacrylic acid and ethyl acrylate copolymers, methacrylic acid and methyl methacrylate copolymers (1:1 ratio), methacrylic acid and methyl methacrylate copolymers (1:2 ratio), Polyvinyl acetate phthalate (PVAP) and Shellac resins. Particularly preferred polymers include shellac, anionic copolymers based on methyl acrylate, methyl methacrylate and methacrylic acid, and methacrylic acid and methyl methacrylate copolymers (1:2 ratio). Ideally, the polymer dissolves at a pH equal to 6.0 and above, preferably 6.5 and above. Suitable coatings may also be obtained by mixing the polymers and copolymers aforementioned.

In a particular embodiment, the formulation comprises a further intermediate coating located between the core and the external pH-dependent layer. The intermediate coating can be formed from a variety of polymers, including pH-dependent polymers, pH-independent water soluble polymers, pH-independent insoluble polymers, and mixtures thereof. Examples of such pH-dependent polymers include shellac type polymers, anionic copolymers based on methylacrylate, methylmethacrylate and methacrylic acid, methacrylic acid and ethyl acrylate copolymers, hydroxypropyl methylcellulose phthalate (HPMCP), and hydroxypropylmethylcellulose acetate succinate (HPMCAS). Examples of pH-independent water soluble polymers include PVP or high molecular weight cellulose polymers such as hydroxypropylmethylcellulose (HPMC) or hydroxypropylcellulose (HPC). Examples of pH-independent insoluble polymers include ethylcellulose polymers or ethyl acrylate and methyl methacrylate copolymers.

In a particular embodiment, the invention uses a formulation comprising:
a core comprising a mixture of an adsorbent (preferably activated charcoal) with carrageenan (preferably kappa-carrageenan),
a intermediate coating selected in the group consisting of HPMC, ethylcellulose and a mixture of methacrylic acid and ethyl acrylate copolymer such as Eudragit® L30D-55, and ethyl acrylate and methyl methacrylate copolymer such as Eudragit® NE30D (for example in a mixture ratio of 1:9 to 9:1, preferably of 2:8 to 3:7), and
an external layer of an anionic copolymer based on methyl acrylate, methyl methacrylate and methacrylic acid, such as Eudragit® FS30D.

In a specific embodiment, the formulation comprises a core, comprising 85% activated charcoal, and 15% Gelcarin GP911 (kappa-carrageenan) and a coating with Eudragit® FS30D or Eudragit® L30D55 (Evonik, Darmstadt, Germany).

### Method of Use

The present invention relates to compositions and methods for treating or preventing a Clostridium-associated disease, or for suppressing pathogenicity of a Clostridium bacterium, based on the use of adsorbents. Adsorbents have been used in the art to reduce or avoid side effects of some active compounds, by sequestering remaining amounts of said active compounds in the intestine. For instance, adsorbents with a controlled release formulation administered together with antibiotics can sequester antibiotics remaining in the lower part of the intestine, thereby avoiding inappropriate antibiotic-induced dysbiosis, generation of antibiotic-resistant bacteria and related or consequential disorders. Surprisingly, by conducting further testing, the inventors have now discovered that adsorbents can have an effect on the very pathogenicity of Clostridium bacteria. As illustrated in the examples, Clostridium bacteria cultured in a medium exposed to adsorbents and Clostridium bacteria exposed to adsorbents, for example during culture, lose their virulence as measured by a potent suppression of toxin production and/or release, sporulation and germination. The results are particularly remarkable since they demonstrate the ability to suppress the pathogenicity of Clostridium difficile, some strains of which are particularly virulent.

The invention thus relates to methods for suppressing the virulence of Clostridium bacteria by exposing such bacteria or their direct environment to an adsorbent in vitro, ex vivo or in vivo.

The invention also relates to methods for treating a Clostridium-associated disease by administering an adsorbent to a mammal.

The invention also relates to methods for preventing a Clostridium-associated disease by administering an adsorbent to a mammal.

Clostridium-associated diseases designate any condition or disease caused by or linked to an infection or colonization with a Clostridium bacterium. Diseases associated with infection by a Clostridium bacterium such as C. difficile can be mild to life-threatening. Disorders of mild cases include watery diarrhea, three or more times a day for several days, abdominal pain or tenderness. Disorders of more severe C. difficile infection include watery diarrhea, up to 15 times each day, severe abdominal pain, loss of appetite, fever, blood or pus in the stool, or weight loss.

The invention particularly relates to a method for suppressing or delaying the production of toxins by a bacterium of the genus Clostridium, comprising exposing such bacteria to an adsorbent in vitro, ex vivo or in vivo.

The invention particularly relates to a method for suppressing or delaying the production of toxins by a bacterium of the genus Clostridium, comprising exposing the environment of such bacteria, for example its growth environment, to an adsorbent in vitro, ex vivo or in vivo.

The invention also relates to a method for suppressing or delaying the germination of spores of a bacterium of the genus Clostridium, comprising exposing such bacteria to an adsorbent in vitro, ex vivo or in vivo.

The invention also relates to a method for suppressing or delaying the germination of spores of a bacterium of the genus Clostridium, comprising exposing the environment of such bacteria, for example its growth environment, to an adsorbent in vitro, ex vivo or in vivo.

The invention also relates to a method for suppressing or delaying the sporulation of a bacterium of the genus Clostridium, comprising exposing such bacteria to an adsorbent in vitro, ex vivo or in vivo.

The invention also relates to a method for suppressing or delaying the sporulation of a bacterium of the genus Clostridium, comprising exposing the environment of such bacteria, for example its growth environment, to an adsorbent in vitro, ex vivo or in vivo.

The term “treatment” designates within the context of the invention either a curative or a preventive treatment of a condition or infection. The term curative indicates particularly the treatment of an existing condition, and includes a suppression, reduction or delay of the extent, development, severity, morbidity and/or duration of the condition or of its symptoms. The term “preventive” designates the treatment of a subject prior to occurrence of a disease or infection, in order to protect the subject from development of a Clostridium-associated disease. The term treatment particularly includes treating a subject having a Clostridium-associated disease, in order for example to suppress the disease or reduce its extent, duration, severity, morbidity or symptoms. The term treatment also includes treating a subject exposed to Clostridium bacteria, or at risk of infection by Clostridium, in order to inhibit or reduce or avoid development of a Clostridium-associated disease in said subject. The invention may also be used to prevent or reduce dissemination of Clostridium spores and/or Clostridium-associated disorders.

The term “environment” designates within the context of the invention the environment of growth of the bacterium. It can refer to a culture medium or growth medium when the bacterium is cultured in vivo. It can further refer to a mammal body fluid, such as intestinal fluid or colonic fluid, where the bacterium is present and can grow and multiply. It can further refer to any compartment of the human body that Clostridium bacteria may colonize.

The term “suppressing pathogenicity” or “suppressing virulence” designates at least a reduction of the pathogenicity/virulence of a bacterium. Preferably the term indicates a substantial reduction by at least 20%, even more preferably at least 50% or more of the pathogenicity/virulence, as measured for instance by the release of toxins, the sporulation or spore germination. As shown in the examples, treatment according to the invention can suppress to below detection levels toxin release from Clostridium, and can reduce by 2 orders of magnitude (i.e. 100-fold) or more the sporulation or spore germination, thus rendering less pathogenic and even non-pathogenic such bacteria.

An object of the present invention thus resides in a method of suppressing the pathogenicity or virulence of a Clostridium bacterium, comprising exposing said bacterium, or their direct environment, in vitro, ex vivo or in vivo to an adsorbent. Such a method may be used in laboratory or in any environment, especially in vivo, to control the virulence of Clostridium bacteria. Such a method can also be used in vivo in a mammal to avoid Clostridium-associated diseases.

In a particular embodiment, suppression of the pathogenicity or virulence is provided by slowing-down the development of the Clostridium bacterium. In another embodiment, suppression of the pathogenicity or virulence is provided by slowing-down the multiplication of the Clostridium bacterium. In a further embodiment, suppression of the pathogenicity or virulence is provided by slowing-down the progression of a Clostridium bacterium. In another embodiment, suppression of the pathogenicity or virulence is provided by slowing-down the life cycle of a Clostridium bacterium. In another embodiment, suppression of the pathogenicity or virulence is provided by a slow-down of the evolution of the infection. In a further embodiment, suppression of the pathogenicity or virulence is provided by a slow-down of the evolution of the symptoms of the infection.

The invention thus also relates to a method for slowing-down:
the development of a Clostridium bacterium,
the multiplication of a Clostridium bacterium,
progression of a Clostridium bacterium,
life cycle of a Clostridium bacterium,
the evolution of an infection by a Clostridium bacterium, or
the evolution of the symptoms of an infection by a Clostridium bacterium,
   comprising administering to a subject in need thereof an effective amount of an adsorbent.

The invention also relates to an adsorbent for use in a method for slowing-down:
the development of a Clostridium bacterium,
the multiplication of a Clostridium bacterium,
progression of a Clostridium bacterium,
life cycle of a Clostridium bacterium,
the evolution of an infection by a Clostridium bacterium, or
the evolution of the symptoms of an infection by a Clostridium bacterium,
   comprising administering to a subject in need thereof an effective amount of an adsorbent.

An object of the present invention thus also relates to a method for slowing the development of a Clostridium bacteria. Thanks to this method, the development of the infection caused by the bacteria is slowed-down, which is advantageous in several aspects, in particular for giving more time to the immune system and/or medical care to combat the infection. The method of the invention provides also more time to the intestinal microbiota to reorganize and reject the Clostridium bacteria. The slowed development of the bacteria may result in an alleviation of the symptoms of the infection or even in a total suppression of the symptoms of the infection.

In the context of the present invention, slowing-down of the Clostridium bacteria may occur in each phase of its cellular life and development. The invention thus provides a method for decreasing the number of vegetative bacteria and/or spores present in the gut of a subject. The resulting decrease may also reduce, as a consequence, the number of bacteria and/or spores potentially excreted by the subject and contaminating the environment, that could potentially provoke further onsets of the disease in other subjects sharing the same environment.

In one embodiment of the invention, the adsorbent is administered independently of any other treatment. An object of the invention is thus to prevent the growth and/or progression through the life cycle of Clostridium bacteria even in the absence of any induced disruption of the microbiota of the patient. Thus, in a particular aspect, the invention provides a method for preventing or slowing-down the growth and/or progression through the life cycle of a Clostridium bacteria, comprising administering an adsorbent to a subject in need thereof, wherein the patient is not concomitantly treated with an antibiotic. In the context of the present invention, the expression “the patient is not concomitantly treated with an antibiotic” refers to a patient that is not administered with an antibiotic the day the adsorbent is administered to him/her and optionally the day before the adsorbent is administered. In another embodiment, in addition to the absence of administration of an antibiotic the same day as the administration of the adsorbent, no antibiotic treatment was administered to the patient from 1 to 30 days before the administration of the adsorbent, such as from 1 to 25, such as from 1 to 20, such as from 1 to 15, such as from 1 to 10, such as from 1 to 9, from 1 to 8, from 1 to 7, from 1 to 6, from 1 to 5, from 1 to 4, from 1 to 3 or from 1 to 2 days before the administration of the adsorbent. In another embodiment, in addition to the absence of administration of an antibiotic the same day as the administration of the adsorbent, no antibiotic treatment was administered to the patient from 1 to 30 days after the administration of the adsorbent, such as from 1 to 25, such as from 1 to 20, such as from 1 to 15, such as from 1 to 10, such as from 1 to 9, from 1 to 8, from 1 to 7, from 1 to 6, from 1 to 5, from 1 to 4, from 1 to 3 or from 1 to 2 days after the administration of the adsorbent.

An object of the present invention also resides in a method of suppressing the pathogenicity or virulence of a Clostridium bacterium, comprising exposing the environment of said bacterium in vitro, ex vivo or in vivo to an adsorbent. Such a method may be used in laboratory or in any environment, especially in vivo, to control the virulence of Clostridium bacteria. Such a method can also be used in vivo in a mammal to avoid Clostridium-associated diseases.

In a particular embodiment, the adsorbent is administered preventively to subjects during an outbreak of Clostridium difficile, to prevent the spread of the disease and reduce the burden or severity or morbidity or mortality of the CDAD if the subject is affected by the outbreak i.e. infected by Clostridium.

In another particular embodiment, the adsorbent is administered to subjects after an episode of Clostridium difficile infection, to prevent the sporulation and shedding of spores in the environment of the patient, thus decreasing the risk of further infections in the environment of the patient and preventing outbreaks in healthcare facilities, including hospitals, nursing homes and the like, or in any other life environment.

In another embodiment, the adsorbent is administered to subjects in order to decrease the severity of a Clostridium difficile infection.

In another embodiment, the adsorbent is administered to subjects in order to speed up the recovery of the patient after a Clostridium difficile infection.

In another embodiment, the adsorbent is administered to subjects in order to prevent the recurrence of a Clostridium difficile infection, i.e. the occurrence of a novel Clostridium difficile infection episode by the same or another strain, in a period up to 90 days after the end of a previous Clostridium difficile infection episode.

The invention may also be used to treat subjects, for example in healthcare facilities, during and/or after an outbreak of Clostridium difficile, to prevent the sporulation and shedding of spores and reduce the overall severity of the outbreak.

The invention can also be used to treat subjects after a Clostridium difficile infection, in order to hasten recovery and reduce the chances of recurrence by tampering with the pathogenicity of Clostridium difficile.

The invention is also particularly suited to treat subjects at risk of contracting Clostridium difficile, for example medical care staff in contact with patients with CDAD, or family members of patient affected by CDAD and/or infected by Clostridium.

The invention can also be used appropriately in patients at risk of CDAD such as patients taking antibiotics, patients above 50 or preferably above 65, or even more preferably above 75, patients with a recent history of hospitalizations to prevent CDAD occurrence or reduce the severity of a CDAD episode should one episode occur despite the initial treatment with the invention.

The invention can further be used in patients at high risk of CDAD such as patients who had a previous episode of CDAD in the years prior to a novel antibiotic cure, a novel hospitalization or a novel immune-suppressive cure.

The invention can further be used in patients already colonized by Clostridium, notably Clostridium difficile, prevent any onset of CDAD and to reduce the pathogenicity of the bacterium.

Further aspects and advantages of the invention will be disclosed in the following illustrative experimental section.

### EXAMPLES

### Example 1: Activated Charcoal Delays and Suppresses the Sporulation and Production of Toxins by C. difficile in BHI Broth

5 mL of BHI (Brain Heart Infusion) broth is incubated with or without activated charcoal (a pharmaceutical grade activated charcoal of vegetal origin at the concentration of 0.05 g/mL) for 2 hours, whereas 5 mL of BHI broth is incubated for 2 hours without the activated charcoal formulation. A half of each sample is centrifuged and filtered with 0.22 μm filters whereas the other half is not centrifuged and filtered. The centrifugation/filtration with 0.22 μm filters removes the activated charcoal in the samples incubated with activated charcoal.

The table below summarizes the 4 conditions:

| | Centrifugation/filtration | No centrifugation/ |
|---|---|---|
| BHI broth submitted to: | with 0.22 μm filters | filtration |
| 2 hours incubation with | Spun activated charcoal | Activated charcoal |
| activated charcoal | | |
| No contact with | Spun control | Control |
| activated charcoal | | |

In the “Control” group, the culture BHI broth has not been treated.
In the “Spun control” group, the culture broth is only centrifuged/filtered.
In the “Activated charcoal” group, the culture both is incubated for 2 hours with activated charcoal and the activated charcoal is still present in the samples.
In the “Spun activated charcoal” broth, the culture both is incubated for 2 hours with activated charcoal and the activated charcoal is removed from the samples by centrifugation/filtration.

All the broths are then pre-reduced overnight, to remove dissolved oxygen and provide anaerobic culture conditions, before C. difficile inoculation.

C. difficile is grown anaerobically on CCEYL agar or Columbia blood agar for 48 hours. Growth from the plate is inoculated into pre-reduced broths treated as described above and incubated anaerobically at 37° C. After 48 hours, 1 mL aliquots are centrifuged and the supernatants removed for toxin testing. In addition, aliquots (500 μL) are taken for vegetative bacteria and spore enumeration. After 72 hours, additional aliquots are taken for sporulation analysis. Vegetative bacteria and spore populations are enumerated at 48 and 72 hour by inoculation of CCEYL agar and phase microscopy.

Toxin testing is performed as follows. Samples for testing are diluted (10-fold series) in PBS. Neat (i.e. culture supernatants) and diluted samples are added to a Vero cell monolayer in duplicate. Specificity of action is determined by neutralization with C. sordellii anti-toxin. Culture supernatant of C. difficile ribotype 027 in BHI broth is used as a positive technical control (toxin titre ˜4 RU). After 48 hours, cell rounding is microscopically determined. Rounding observed in 80% of the monolayer is considered positive. A positive neat sample is given a titre of 1, a positive 1:10 diluted sample is given a titre of 2 and so on. Samples are repeated on 2 separate monolayers (technical repeats).

Agar enumeration is performed as follows. Samples are diluted (10-fold dilution series) in peptone water (with and without alcohol shock) and C. difficile total and spore counts (following alcohol shock) enumerated on Brazier's CCEYL agar. Each dilution (20 μL) is plated onto one quarter agar plate and colonies counted (between 20-100 cfu/dilution) after 48 hours

Phase microscopy is performed as follows. 70 μL culture fluid is placed on microscopy slide and heat fixed at 50° C. The sample is overlaid with 70 μL molten Wilkins-Chalgren agar and a cover slip. Once the agar is dried, the percentage phase bright spores, phase dark spores and vegetative bacteria is determined (100 entities counted from at least 3 different fields of view in triplicate).

The process described above is repeated for 3 strains of C. difficile known as ribotypes 001, 027 and 078.

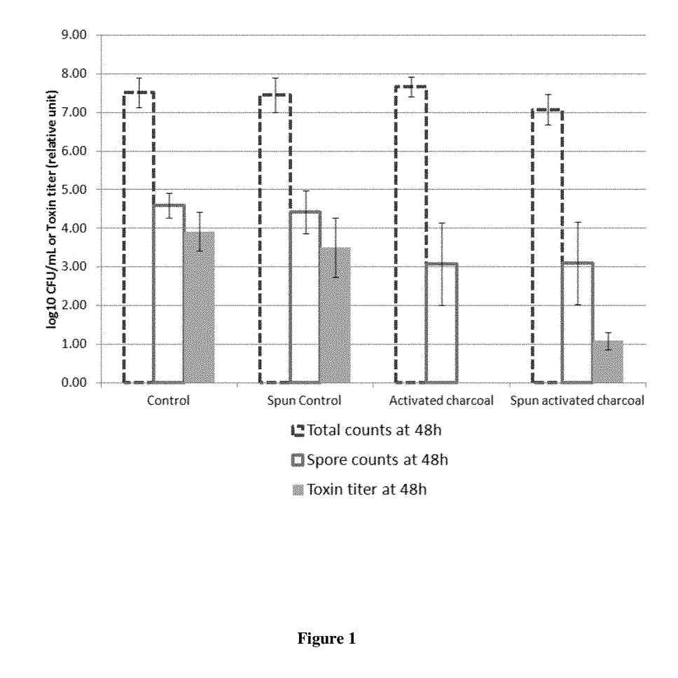

FIG. 1 shows the counts of C. difficile bacteria and spores after 48 h of culture in BHI broth treated or not with activated charcoal as explained above. FIG. 1 also presents the toxin titers in each sample after 48 h of culture.

FIG. 1 shows that treatment of BHI broth with activated charcoal effectively suppressed the pathogenicity of C. difficile bacteria. More particularly, in the 2 groups where the broth was pre-treated with activated charcoal, a substantial decrease of at least one log in the counts of spores (i.e. >90% of reduction) was observed. Furthermore, in the 2 groups where the broth was pre-treated with activated charcoal, a very marked decrease of the toxin titers (with more than 2 units of difference, i.e. at least 100-fold) was measured as early as after 48 hours after treatment. Remarkably, such an effect was observed also in the spun activated charcoal group, where exposure of the broth to activated charcoal was only transient, prior to inoculation with Clostridium difficile bacteria. These results show that exposure of the culture medium to activated charcoal delayed or suppressed the sporulation of and toxin production and release by Clostridium difficile, demonstrating a very marked loss of virulence of Clostridium difficile.

### Example 2: Activated Charcoal Delays and Suppresses the Sporulation and Production of Toxins by C. difficile in Fecal Slurry Broth

A 10% slurry of pooled faeces from healthy donors is made in pre-reduced gut model broth as described in Freeman, J., O'Neill, F. J., and Wilcox, M. H. Effects of cefotaxime and desacetylcefotaxime upon Clostridium difficile proliferation and toxin production in a triple-stage chemostat model of the human gut. Journal of Antimicrobial Chemotherapy 2003; 52: 96-102.

5 mL of this fecal slurry broth is incubated with or without activated charcoal (a pharmaceutical grade activated charcoal of vegetal origin at the concentration of 0.05 g/mL) for 2 hours. Half of each sample is centrifuged and filtered with 0.22 μm filters whereas the other half is not centrifuged and filtered. The centrifugation/filtration with 0.22 μm filters removes the activated charcoal in the samples incubated with activated charcoal.

The table below summarizes the 4 conditions:

| Fecal slurry broth | Centrifugation/filtration | No centrifugation/ |
|---|---|---|
| submitted to: | with 0.22 μm filters | filtration |
| 2 hours incubation with | Spun activated charcoal | Activated charcoal |
| activated charcoal | | |
| No contact with | Spun control | Control |
| activated charcoal | | |

In the “Control” group, the culture fecal slurry broth has not been treated.
In the “Spun control” group, the culture broth is only centrifuged/filtered.
In the “Activated charcoal” group, the culture both is incubated for 2 hours with activated charcoal and the activated charcoal is still present in the samples.
In the “Spun activated charcoal” broth, the culture both is incubated for 2 hours with activated charcoal and the activated charcoal is removed from the samples by centrifugation/filtration.

The experiment is conducted with the modus operandi presented in example 1.

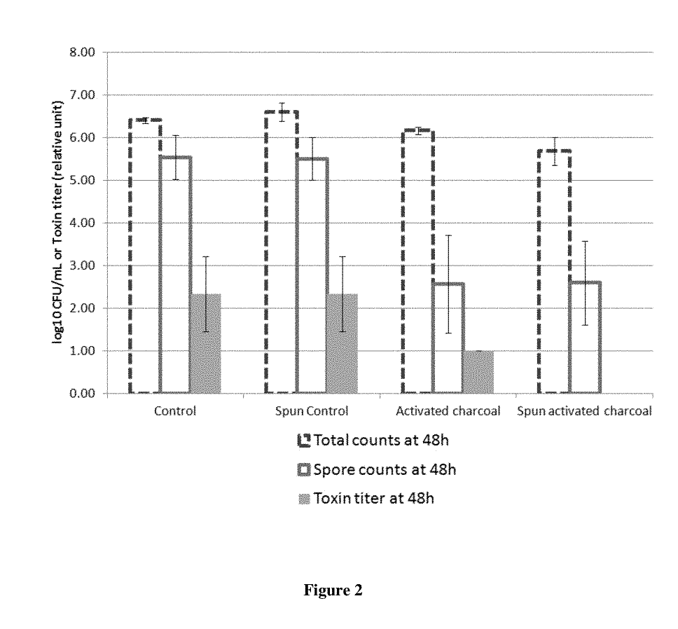

FIG. 2 shows the counts of C. difficile bacteria and spores after 48 h of culture in fecal slurry broth treated or not with activated charcoal as described above. FIG. 2 also presents the toxin titers in each sample after 48 h of culture.

FIG. 2 shows that treatment of fecal slurry broth with activated charcoal effectively suppressed the pathogenicity of C. difficile bacteria. More particularly, in the 2 groups where the fecal slurry broth was pre-treated with activated charcoal, a substantial decrease of at least 2 logs in the counts of spores (i.e. >99% of reduction) was observed after 48 h of culture. Furthermore, in the 2 groups where the fecal slurry broth was pre-treated with activated charcoal, a very marked decrease of the toxin titers (with more than 1 unit difference, i.e. at least 10-fold) was measured as early as 48 hours after treatment. Remarkably, such effect was observed also in the spun activated charcoal group, where exposure of the fecal slurry broth to activated charcoal was only transient prior to inoculation with Clostridium difficile bacteria. These results show that exposure of the fecal slurry broth to activated charcoal delayed or suppressed the sporulation of and toxin release by Clostridium difficile, demonstrating a very marked loss of virulence of Clostridium difficile.

### Example 3: Activated Charcoal Delays or Suppresses the Sporulation and Production of Toxins by C. difficile in Gut Model Broth

5 mL of pre-reduced gut model broth (GM broth) (Freeman, J., O'Neill, F. J., and Wilcox, M. H. Effects of cefotaxime and desacetylcefotaxime upon Clostridium difficile proliferation and toxin production in a triple-stage chemostat model of the human gut. Journal of Antimicrobial Chemotherapy 2003; 52: 96-102) is incubated with or without activated charcoal (a pharmaceutical grade activated charcoal of vegetal origin at the concentration of 0.05 g/mL) for 2 hours. Half of each sample is centrifuged and filtered with 0.22 μm filters whereas the other half is not centrifuged and filtered. The centrifugation/filtration with 0.22 μm filters removes the activated charcoal in the samples incubated with activated charcoal.

The table below summarizes the 4 conditions:

| Gut model broth | Centrifugation/filtration | No centrifugation/ |
|---|---|---|
| submitted to: | with 0.22 μm filters | filtration |
| 2 hours incubation with | Spun activated charcoal | Activated charcoal |
| activated charcoal | | |
| No contact with | Spun control | Control |
| activated charcoal | | |

In the “Control” group, the culture GM broth has not been treated.
In the “Spun control” group, the culture broth is only centrifuged/filtered.
In the “Activated charcoal” group, the culture both is incubated for 2 hours with activated charcoal and the activated charcoal is still present in the samples.
In the “Spun activated charcoal” broth, the culture both is incubated for 2 hours with activated charcoal and the activated charcoal is removed from the samples by centrifugation/filtration.

The experiment is conducted with the modus operandi presented in example 1.

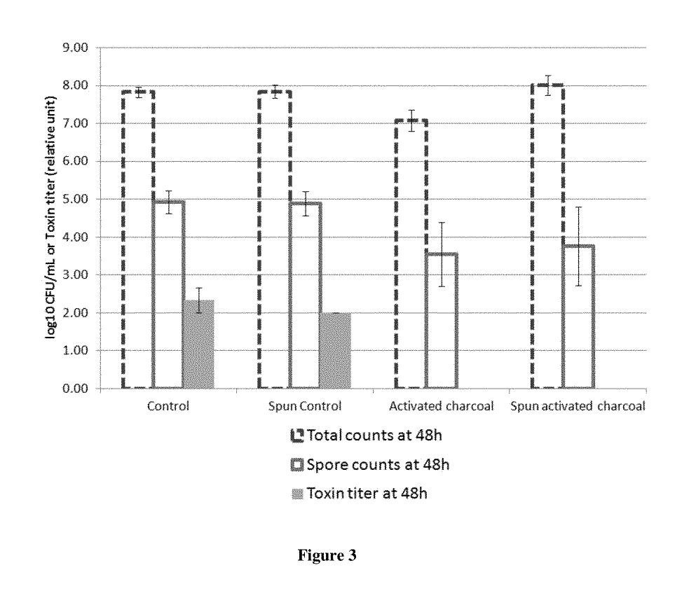

FIG. 3 shows the counts of C. difficile bacteria and spores after 48 h of culture in the gut model broth treated or not with activated charcoal as described above. FIG. 3 also presents the toxin titers in each sample after 48 h of culture.

FIG. 3 shows that treatment with activated charcoal effectively suppressed the pathogenicity of C. difficile bacteria. More particularly, in the 2 groups where the GM broth was pre-treated with activated charcoal, a substantial decrease of at least 1 log in the counts of spores (i.e. >90% of reduction) was observed. Furthermore, in the 2 groups where the GM was pre-treated with activated charcoal, a very marked decrease of the toxin titers (with more than 2 units difference, i.e. at least 100-fold) was measured after 48 h of culture. Remarkably, such effect was observed also in the spun activated charcoal group, where exposure to activated charcoal was only transient, prior to inoculation with Clostridium difficile bacteria. These results show that exposure of the GM to activated charcoal delayed or suppressed the sporulation of and toxin release by Clostridium difficile, demonstrating a very marked loss of virulence of Clostridium difficile.

### Example 4: Activated Charcoal Delays or Suppresses the Production of Toxins in BHI Broth for 3 Different Strains of C. difficile

Aliquots of BHI medium are prepared and pre-reduced. Activated charcoal (a pharmaceutical grade activated charcoal of vegetal origin at the concentration of 0.05 g/mL) is added to the growth medium at different time points: 3 hours before C. difficile inoculation; at the same time as C. difficile inoculation; or 3, 6 hours after inoculation. Toxin titers were then measured at 6, 24 and 48 hours post C. difficile inoculation. No activated charcoal is incubated in the growth medium of the control group.

The growth, inoculation and measure of toxins titer are conducted with the modus operandi presented in example 1.

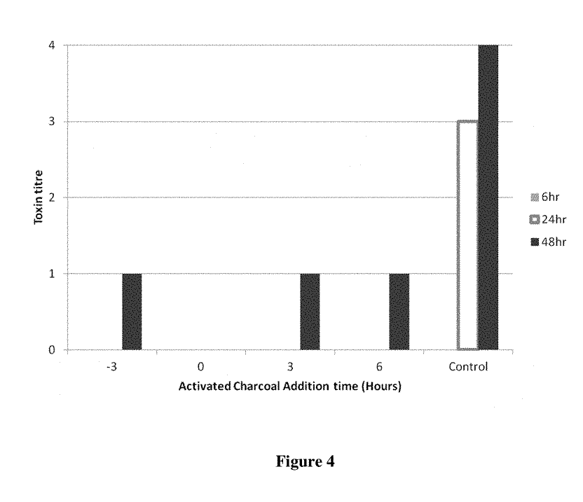

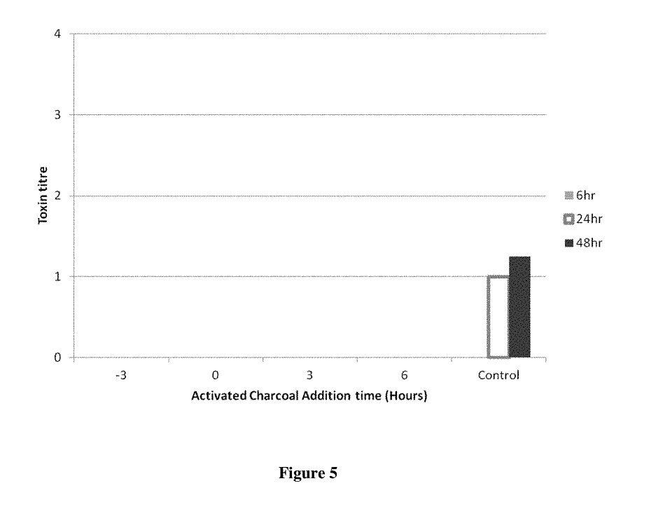

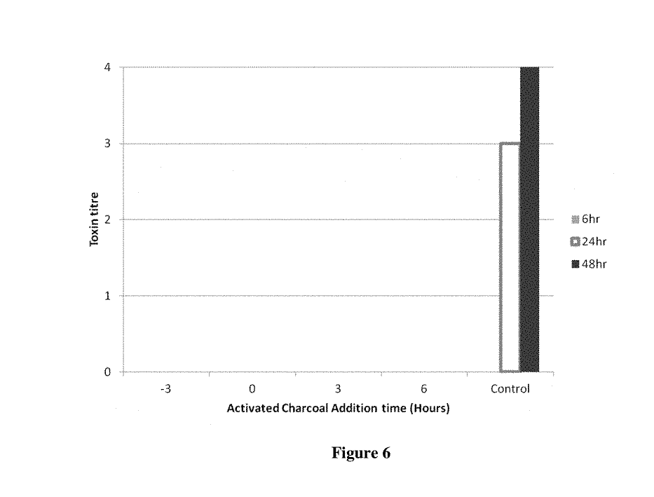

FIGS. 4, 5 and 6 show that the treatment with activated charcoal effectively suppressed the pathogenicity of C. difficile bacteria. More particularly, all of the groups where the broth was treated with activated charcoal show a substantial decrease of at least 1 unit in the titers of toxins and up to 4 units of titers of toxins in difference.

These data show that pre-exposure of the environment to an activated charcoal effectively prevented colonization or virulence of Clostridium bacteria. Particularly, when the medium is exposed to activated charcoal 3 hours before inoculation of the bacteria, essentially no toxins are detected in the medium even 48 hours after inoculation showing that the inoculated bacteria were rendered non pathogenic.

Similarly, when the medium is exposed to activated charcoal at the same time or within the first 6 hours after inoculation of the bacteria, essentially no toxins are detected in the medium, even 48 hours after inoculation. These results further confirm that activated charcoal can effectively treat a Clostridium-associated disease.

### Example 5: Activated Charcoal Delays or Suppresses the Germination of Spores in BHI Broth for 3 Different Strains of C. difficile

8 mL of BHI broth is incubated with or without activated charcoal (a pharmaceutical grade activated charcoal of vegetal origin at the concentration of 0.05 g/mL) for 2 hours (Spun AC group), whereas 8 mL of BHI broth is incubated for 2 hours without the activated charcoal formulation (Control group). The samples incubated with activated charcoal are then centrifuged and filtered with 0.22 μm filters. The centrifugation/filtration with 0.22 μm filters removes the activated charcoal in the samples incubated with activated charcoal.

A C. difficile spore preparation (˜10⁷ CFU/mL) is inoculated into each aliquot and incubated anaerobically at 37° C. At 24 and 48 hours, 500 μL samples are taken and vegetative bacteria and spore are enumerated by agar and phase microscopy. The agar and phase microscopy enumerations are conducted with the modus operandi presented in example 1.

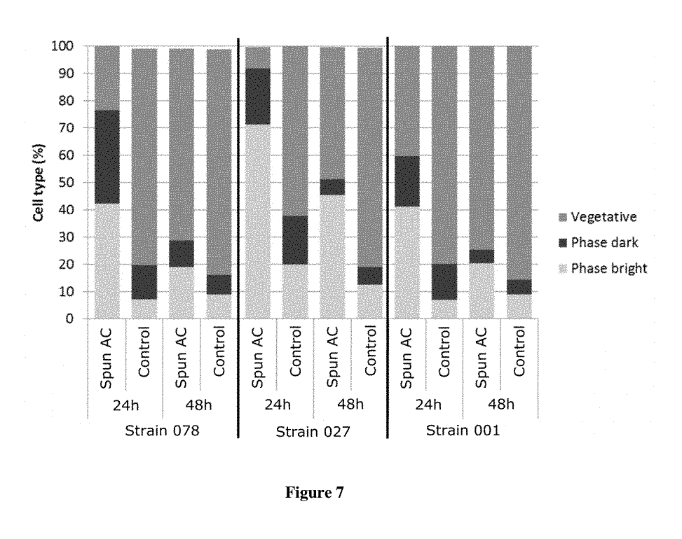

FIG. 7 shows the physiological status of C. difficile spores after 24 h and 48 h of culture. Phase bright cells correspond to spores that have not yet germinated. Phase dark cells correspond to spores during germination. Vegetative cells correspond to mature bacteria after germination of spores.

As shown in FIG. 7, the number of vegetative cells is lower in the groups where the broth was treated with activated charcoal for the 3 strains of C. difficile both at 24 h and 48 h after inoculation with spores. Accordingly, the share of the bacteria population in phase bright state (i.e. spores not yet germinated) is higher in the groups where the broth was treated with activated charcoal prior to inoculation.

These data show that pre-exposure of the environment to an activated charcoal effectively delayed the germination of spores of Clostridium bacteria.

1. An adsorbent for use to suppress or reduce the pathogenicity or virulence of a bacterium of the genus Clostridium.

2. The adsorbent of claim 1, for use to suppress or delay the production of toxins by a bacterium of the genus Clostridium, and/or to suppress or delay the germination of spores of a bacterium of the genus Clostridium, and/or to suppress or delay the sporulation of a bacterium of the genus Clostridium.

3. The adsorbent of claim 1, wherein the adsorbent is activated charcoal.

4. The adsorbent of claim 1, wherein the bacterium of the genus Clostridium is Clostridium difficile.

5. The adsorbent of claim 1, wherein the mammal is a human.

6. The adsorbent of claim 5, wherein the mammal is a human infected with Clostridium or colonized by Clostridium or at risk of infection by Clostridium.

7. The adsorbent of claim 1, wherein the adsorbent is administered preventively to subjects during an outbreak of Clostridium difficile, in order to prevent the spread of the disease.

8. The adsorbent of claim 1, wherein the adsorbent is administered preventively to subjects during an outbreak of Clostridium difficile, in order to reduce the severity and/or burden of the Clostridium difficile infection if the patient is infected during the outbreak.

9. The adsorbent of claim 1, wherein the adsorbent is administered to subjects during and/or after an outbreak of Clostridium difficile, in order to reduce the overall severity of the outbreak.

10. A composition comprising activated charcoal as active agent for use to suppress the pathogenicity of Clostridium difficile in a mammal by delaying or suppressing the production of toxins by Clostridium difficile or by delaying or suppressing the sporulation or the germination of spores of Clostridium difficile.

11. The composition of claim 10, wherein the mammal is a human or a non-human animal infected by Clostridium difficile or at risk of Clostridium difficile infection.

12. The adsorbent of claim 1, wherein the adsorbent is mixed with carrageenan.

13. The adsorbent of claim 12, wherein the adsorbent is in a formulation comprising:
a core containing the adsorbent mixed with carrageenan, and
a layer of external coating formed around the core such that the adsorbent is released from the formulation in the lower part of the small intestine.

14. The adsorbent of claim 2, wherein the adsorbent is activated charcoal.

15. The adsorbent of claim 2, wherein the bacterium of the genus Clostridium is Clostridium difficile.

16. The adsorbent of claim 3, wherein the bacterium of the genus Clostridium is Clostridium difficile.

17. The composition of claim 10, wherein the composition is mixed with carrageenan.

18. The composition of claim 17, wherein the composition is in a formulation comprising:
a core containing the composition mixed with carrageenan, and
a layer of external coating formed around the core such that the composition is released from the formulation in the lower part of the small intestine.
